(19) United States
(12) Patent Application Publication
(10) Pub. No.: US 2024/0207568 A1
(43) Pub. Date: Jun. 27, 2024

(54) Vehicle-mounted aromatherapy device

(71) Applicant: Kai Chen, Shenzhen (CN)

(72) Inventors: Kai CHEN, Shenzhen (CN); Fan YANG, Shenzhen (CN)

(21) Appl. No.: 18/069,329

(22) Filed: Dec. 21, 2022

(51) Int. Cl. A61M 21/02; B60H 3/00

(52) U.S. Cl. CPC A61M 21/02; B60H 3/0028; A61M 2021/0016

(57) ABSTRACT

Disclosed is a vehicle-mounted aromatherapy device. An accommodating cavity is provided inside a main body; a first ventilation structure in communication with the accommodating cavity is provided at one end of the main body, and an air inlet in communication with the accommodating cavity is provided at the other end of the main body; a fragrance device is mounted inside the accommodating cavity of the main body; the fan blade is rotatably connected to the main body; and the fixing member is fixedly connected to the main body. When the vehicle-mounted aromatherapy device is in use, the air flow of an air outlet of an automobile air conditioner is used to accelerate the volatilization of a solid aromatherapy, the aromatherapy substance can be conveniently replaced, and the aromatherapy device can be used for many times.

### TECHNICAL FIELD

The present invention relates to the field of aromatherapy devices, and in particular, to a vehicle-mounted aromatherapy device.

### BACKGROUND

Some automotive perfumes are usually placed in automobiles to remove peculiar smell inside the automobiles, and the fragrance released by the automotive perfumes can keep people clear headed, have a gentle mood, and is deeply loved by car owners. However, some liquid perfumes may spill out when being used due to the vibration of the automobiles, causing pollution in the automobiles, and dangerous conditions such as burning easily in case of cigarette butts or open flames. In addition, the air temperature in summer is relatively high, the liquid perfumes contain flammable components, and is easy to cause safety hazards when placed in the automobiles. Some solid aromatherapy devices have a low volatilization speed, and compared with liquid perfumes, will not pollute the automobile interior trim, but the effect of diffusing fragrance is not ideal. Moreover, most of the existing vehicle-mounted aromatherapy devices are disposable products that do not support the replacement of aromatherapy substances, resulting in waste of resources.

### SUMMARY

An objective of the present invention is to provide a vehicle-mounted aromatherapy device. The vehicle-mounted aromatherapy device is mounted at an air outlet of an air conditioner of an automobile when in use, so that when in use, the air flow of the air outlet of the automobile air conditioner is used to accelerate the volatilization of a solid aromatherapy, and the volatilization of the solid aromatherapy is further accelerated by rotating fan blade. The vehicle-mounted aromatherapy device of the present invention can conveniently replace an aromatherapy substance, and can be used for many times.

In order to achieve the described objective, the present invention provides the following technical solutions:

A vehicle-mounted aromatherapy device, comprising a main body, wherein an accommodating cavity is provided inside the main body, the main body comprises a first ventilation structure and a second ventilation structure, the second ventilation structure is provided at one side of the accommodating cavity and is correspondingly mounted at an air outlet of an indoor air conditioner for air inlet, the first ventilation structure is provided on the other side of the accommodating cavity, the first ventilation structure is used for air outflow of the vehicle-mounted aromatherapy device, and the first ventilation structure and the second ventilation structure are both in communication with the accommodating cavity; a perfume device mounted in the accommodating cavity of the main body; a fan blade mounted at a lateral exterior of the first ventilation structure of the main body, wherein the fan blade is rotatable relative to the main body; and a fixing member fixedly connected to the main body, wherein the fixing member is capable of fixedly connecting the main body to an external object.

Further, the main body comprises a main housing and a front end housing, an air outlet is provided at one end of the main housing, and the front end housing is provided on the air outlet and is detachably connected to the main housing.

Further, an air inlet is provided at the other end of the main housing, the main body further comprises a fixing plate, the fixing plate is mounted on the air inlet of the main housing, and the second ventilation structure is provided on an outer surface of the fixing plate. When the air flows from the second ventilation structure to the first ventilation structure, the fragrance volatilizing out from the perfume inside the main body is diffused, so as to facilitate volatilization of the perfume.

Further, a doll bottom plate is provided on an outer wall of the main housing, and the doll bottom plate is integrally formed with or fixedly connected to the main housing.

Further, two extension plates are symmetrically provided on an inner side of the front end housing, and are used for mounting the perfume device. The perfume device is mounted in the front end housing, and the two extension plates facilitate the perfume sheet of the perfume device to be mounted in the front end housing. When the perfume sheet is replaced, only the front end housing needs to be removed for replacement, and the replacement process is more convenient.

Further, a shaft base is axially provided on the front end housing, the fan blade is connected to the shaft base by means of a fixed shaft, and the fan blade is rotatable relative to the fixed shaft.

Further, a first fixed shaft insertion hole is provided in the center of the fan blade, a support mounting groove is provided in the first fixed shaft insertion hole at the rotation center of the fan blade, and a bearing support is mounted inside the support mounting groove; a second fixed shaft insertion hole used for the fixed shaft to be inserted therethrough is provided on the bearing support, a bearing limiting groove is provided in the second fixed shaft insertion hole, and a bearing is mounted inside the bearing limiting groove; and the fixed shaft passes through the bearing support, the bearing and the fan blade in sequence, and is connected to the shaft base. The bearing is mounted inside the fan blade by means of a bearing support, and the rotation of the fan blades is more stable, reducing the shaking of the fan blade, thereby reducing the noise generated by the shaking of the fan blade.

Further, a first fixed shaft insertion hole is provided at the center of the fan blade, and a bearing mounting groove for mounting the bearing is provided in the first fixed shaft insertion hole; and the fixed shaft passes through the bearing and the fan blade in sequence, so as to be connected to the shaft base. The bearing is directly mounted inside the fan blade, thereby reducing the number of parts and reducing mounting steps.

Further, the vehicle-mounted aromatherapy device further comprises a doll provided on an outer wall of the main body, and the doll is integrally formed with or detachably connected to and the main body.

Further, the perfume device comprises a perfume accommodating box, and at least one perfume accommodating chamber is provided in the perfume accommodating box; a fragrance release port is further provided on one end of the perfume accommodating chamber, and is used for releasing the fragrance volatilized by an internal perfume.

Further, a perfume sheet is mounted inside the perfume accommodating chamber, a perfume sheet limiting plate is further provided at the bottom of the perfume accommodating chamber, and correspondingly, a perfume sheet limiting groove fitted with the perfume sheet limiting plate is provided on the perfume sheet. Thus, the mounting of the perfume sheet is more stable, the old perfume sheet is prevented from being removed conveniently due to the dumping or shifting of the perfume sheet in the perfume accommodating chamber, and the limiting placement of the fragrance sheet is more conducive to replacement operations.

Further, the front end housing is integrally formed with the main housing, a material charging port is provided on the side surface of the main housing, the material charging port is opposite to the perfume accommodating chamber in position, and the doll bottom plate is detachably connected to the material charging port.

A first magnet mounting groove is recessed downwards from the surface of the doll bottom plate, a first magnet is fixedly mounted inside the first magnet mounting groove, correspondingly, a doll bottom plate positioning groove is recessed downwards from the upper surface of the perfume accommodating box and is used for accommodating the first magnet mounting groove, a second magnet mounting groove is arranged directly below the doll bottom plate positioning groove, and a second magnet is fixedly mounted inside the second magnet mounting groove. By means of magnetic attraction, the doll bottom plate is fixedly connected to the main body, the doll bottom plate can be removed at any time to replace the perfume sheet, and the removal and mounting operations are convenient, facilitating the replacement of the perfume sheet.

Further, the doll is integrally formed or fixedly mounted on an upper part of the doll bottom plate.

Further, a doll plug is arranged above the doll bottom plate, and correspondingly, an insertion hole fitted with the doll plug is arranged at the bottom of the doll. The doll bottom plate can be adapted to dolls having different shapes, so as to meet different market demands during mass production.

Further, the fixing member comprises a clamping claw, and the clamping claw is fixedly connected to the fixing plate. The clamping claw clamps the main body at an air outlet of an air conditioner of the automobile, so that air can flow through the main body, so as to facilitate the volatilization of the perfume, and the clamping is stable to prevent the vehicle-mounted aromatherapy device from rotating and dropping when being fixed on the automobile.

Further, the clamping claw comprises a fixing sheet, and at least two claw sheets are arranged on an edge of the fixing sheet.

Further, at least one limiting block is provided on the fixing plate, correspondingly, a limiting groove is provided on the fixing sheet and is used to prevent relative rotation between the clamping claw and the fixing plate.

Further, a clamping jaw sleeve is sleeved on the clamping jaw, so as to increase a friction force between the clamping jaw and the external object.

Further, the main body comprises a main housing and a front end housing, an air outlet is provided at one end of the main housing, and the front end housing is provided on the air outlet and is formed integrally with the main housing.

It can be determined through analysis that the present invention discloses a vehicle-mounted aromatherapy device; a main body of an aromatherapy machine in which an aromatherapy device is mounted is fixed at an air outlet of an air conditioner of an automobile by means of a fixing member, the air conditioner of the automobile is used to blow air to diffuse the fragrance into the automobile better, and the mounting is stable, preventing the phenomenon that the vehicle is unstable in fixed conditions such as jolt, sharp acceleration or deceleration. In the present invention, a solid perfume sheet is mounted inside the vehicle-mounted aromatherapy machine, and the perfume sheet can be replaced by removing a front end housing; and the machine body part of the vehicle-mounted aromatherapy machine can be used for a long time, reducing the waste of resources. In the present invention, a material charging port is provided on a side edge of a main housing, a doll bottom plate is connected to the material charging port by means of magnetic attraction, and the doll bottom plate is removed from the material charging port or covered on the material charging port, so as to facilitate the replacement of perfume. A doll is provided on the doll bottom plate, so that the vehicle-mounted aromatherapy machine as a whole has interestingness; when the doll bottom plate is removed from the material charging port, the doll can serve as a point of exertion, facilitating removal or replacement of the doll bottom plate. A perfume accommodating box is mounted in a vehicle-mounted aromatherapy machine, facilitating the stabilization of a perfume sheet; a perfume sheet limiting plate is further provided in the perfume accommodating chamber thereof, and a perfume sheet limiting groove is provided on the corresponding perfume sheet to further define the position of the perfume sheet inside the aromatherapy machine, so that the perfume sheet is fixed and stable, shaking is reduced, and the perfume sheet is easily replaced. The vehicle-mounted aromatherapy machine is further provided with a fan blade for guiding flow, and the fan blade rotates under the action of blowing of an air-conditioner, so that the fragrance volatilized inside the aromatherapy machine is guided, facilitating the diffusion of the fragrance, and making the whole more interesting.

Compared with the prior art, the vehicle-mounted aromatherapy device of the present invention is mounted at an air outlet of an air conditioner of an automobile and is used for the volatilization and diffusion of solid perfume, avoiding the occurrence of the phenomenon of falling due to instability during the travel of the vehicle; and the vehicle-mounted aromatherapy device of the present invention can replace a perfume sheet, instead of a disposable product, reducing the waste of resources; and in the vehicle-mounted aromatherapy device of the present invention has a simple and convenient method for replacing a fragrance sheet and more stable mounting of the fragrance sheet inside, and the fragrance volatilizes more evenly.

### BRIEF DESCRIPTION OF THE DRAWINGS

The accompanying drawings, which form a part of the present invention, are used for providing a further understanding of the present invention. The schematic embodiments and illustrations of the present invention are used for explaining the present invention, and do not form improper limits to the present invention. In which:

FIG. 1 is a three-dimensional view of a first embodiment;

FIG. 2 is a schematic diagram of an explosion structure of a first embodiment;

FIG. 3 is a sectional view of a fan blade of a first embodiment;

FIG. 4 is a structural diagram of a fixed shaft of a first embodiment;

FIG. 5 is a sectional structural diagram of a fixed shaft of a first embodiment;

FIG. 6 is a sectional structural diagram of a bearing support shaft of a first embodiment;

FIG. 7 is a front-side structural diagram of an integrated front end housing of a first embodiment;

FIG. 8 is a rear-side structural diagram of an integrated front end housing of a first embodiment;

FIG. 9 is a sectional structural diagram of mounting of a fan blade and a front end housing of a first embodiment;

FIG. 10 is a rear-side structural diagram of a main body of a first embodiment;

FIG. 11 is a structural diagram of a fixing plate of a first embodiment;

FIG. 12 is a structural diagram of a clamping jaw of a first embodiment;

FIG. 13 is a structural diagram of a doll of a first embodiment;

FIG. 14 is a structural diagram of a detachable structure of a front end housing and a shaft base of a first embodiment;

FIG. 15 is a structural diagram of a fixing connection between a perfume accommodating box and a fixing plate of a second embodiment;

FIG. 16 is a structural diagram of the top of a perfume accommodating box of a second embodiment;

FIG. 17 is a cross-sectional view taken along line A-A in FIG. 16;

FIG. 18 is a structural diagram of a perfume sheet of a second embodiment;

FIG. 19 is a structural diagram of a main body of a third embodiment;

FIG. 20 is a rear-side structural diagram of a main body of a third embodiment;

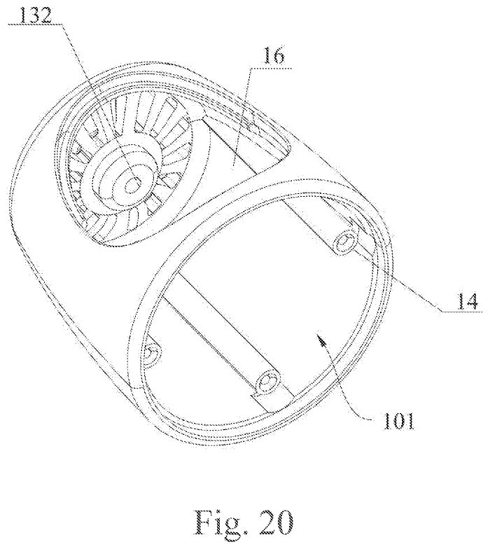

FIG. 21 is a sectional structural diagram of a fan blade of a fourth embodiment;

FIG. 22 is a sectional structural diagram of a fan blade and a main body after assembly of a fourth embodiment;

FIG. 23 is a structural diagram of a doll bottom plate of a fourth embodiment; and

FIG. 24 is a schematic diagram of a mounting explosion structure of a fourth embodiment.

### DESCRIPTION OF REFERENCE NUMERALS

1—main body; 10—main housing; 101—air inlet; 102—accommodating cavity; 103—air outlet; 11—front end housing; 111—extension plate; 112—first ventilation structure; 113—shaft base; 1131—shaft limiting hole; 1132—screw through hole; 14—bolt fixing column; 15—fixing plate; 151—mounting hole; 152—second ventilation structure; 153—limiting block; 16—material charging port; 17—doll bottom plate; 171—doll plug; 172—first magnet mounting groove; 18—first magnet; 2—fragrance device; 20—fragrance sheet; 201—fragrance sheet accommodating groove; 21—fragrance accommodating box; 210—fragrance accommodating chamber; 211—fragrance release port; 212—fragrance sheet limiting plate; 213—doll bottom plate positioning groove; 214—second magnet mounting groove; 22—second magnet; 3—fan blade; 30—first fixed shaft insertion hole; 31—support mounting groove; 32—bearing mounting groove; 4—fixed shaft; 41—limiting shaft segment; 42—screw hole; 5—bearing; 6—bearing support; 60—second fixed shaft insertion hole; 61—bearing limiting groove; 7—fixing member; 71—clamping claw; 711—claw sheet; 712—limiting groove; 72—clamping claw sleeve; 8—doll; and 81—insertion hole.

### DETAILED DESCRIPTION OF THE EMBODIMENTS

The present invention will be described in detail below with reference to the accompanying drawings and in combination with embodiments. The respective examples are provided by means of explanation of the present invention without limiting the present invention. Indeed, it will be apparent to a person skilled in the art that modifications and variations can be made in the present invention without departing from the scope or spirit of the present invention. For example, features illustrated or described as being part of one embodiment can be used in another embodiment to yield yet another embodiment. Therefore, it is expected that the present invention includes such modifications and variations within the scope of the appended claims and their equivalents.

In the description of the present invention, orientation or position relationships indicated by terms such as “longitudinal”, “lateral”, “up”, “down”, “front”, “rear”, “left”, “right”, “vertical”, “horizontal”, “top”, and “bottom” are based on orientation or position relationships shown in the accompanying drawings, which are only used to facilitate description of the present invention rather than requiring that the present invention must be constructed and operated in a specific orientation, and therefore cannot be construed as limitations to the present invention. The terms “connecting”, “connected” and “provided” used in the present invention should be understood broadly, for example, can be a fixed connection or a detachable connection; can also be direct connections or indirect connections by means of intervening components; can also be wired connections or radio connections; and can also be wireless communication signal connections. A person skilled in the art would have been able to understand the specific meaning of the described terms according to specific situations.

One or more examples of the present invention are shown in the accompanying drawings. The detailed description uses numerals and letter labels to refer to features in the drawings. Like or analogous labels in the drawings and descriptions have been used to refer to like or analogous parts of the present invention. As used herein, the terms “first”, “second”, “third”, and the like are used interchangeably to distinguish one member from another, and are not intended to represent the location or importance of the individual members.

### First Embodiment

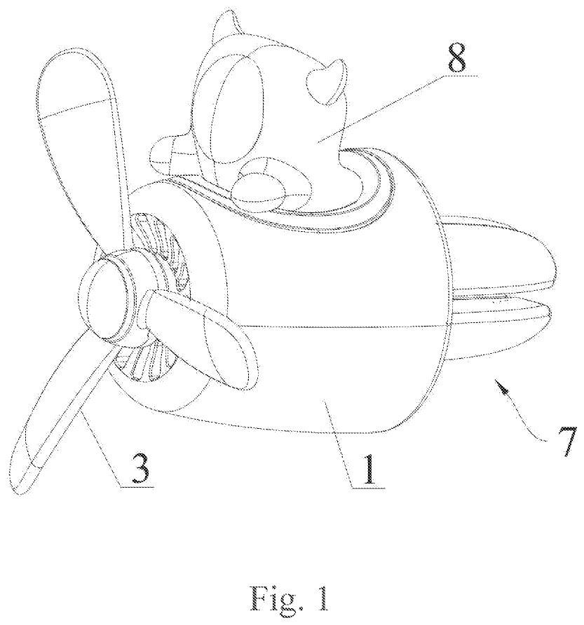

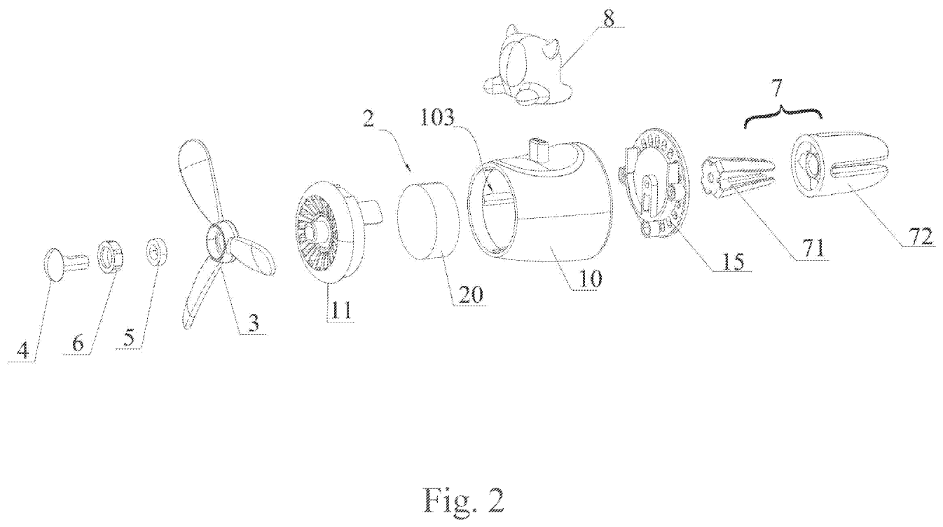

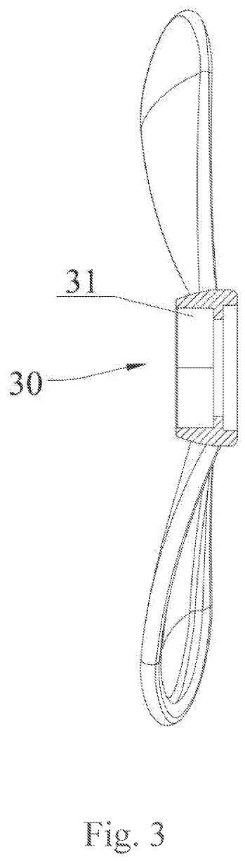

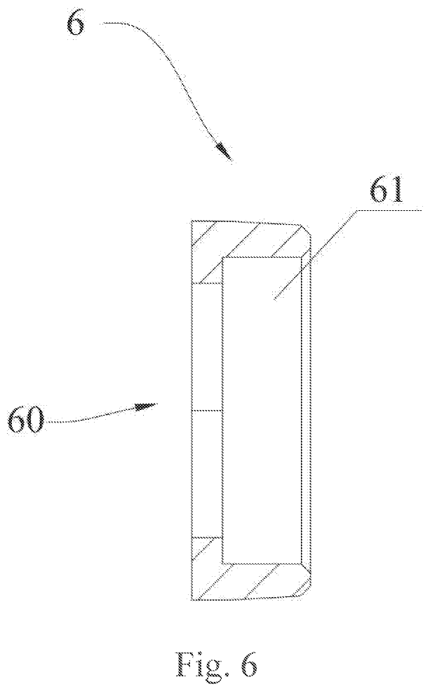

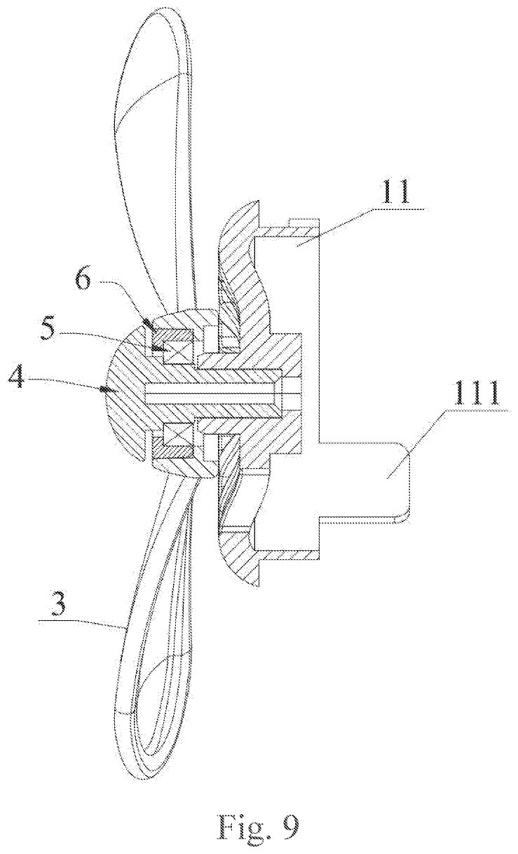

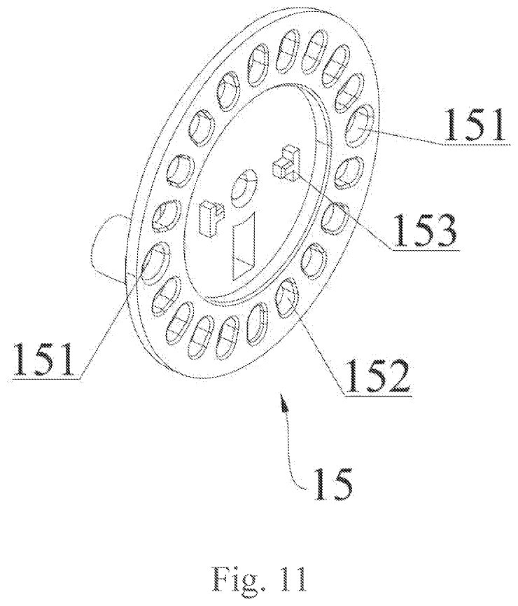

As shown in FIG. 1 to FIG. 14, according to the embodiments of the present invention, an vehicle-mounted aromatherapy device is provided, comprising a main body 1; an accommodating cavity 102 is provided inside the main body 1, and a perfume device 2 is provided in the accommodating cavity 102; the main body 1 comprises a main housing 10, the main housing 10 is a hollow housing having the accommodating cavity 102 therein, and an air outlet 103 is provided on one end of the main housing 10; a front end housing 11 is provided at the air outlet 103, and the front end housing 11 and the main housing 10 are detachably mounted; and a first ventilation structure 112 in communication with an accommodating cavity 102 of the main housing 10 is provided on the front end housing 11. An air inlet 101 is provided at the rear end of the main body 10, and the air inlet 101 is connected to the first ventilation structure 112 so as to form an air circulation channel inside the main body 1. A fan blade 3 for air guiding is further mounted on the front end housing 11, and a fixed shaft 4 is mounted inside the fan blade 3 in a penetrating manner, a bearing 5 is sleeved on the fixed shaft 4, and the fan blade 3 rotates relative to the fixed shaft 4 by means of the bearing 5. Specifically, as shown in FIGS. 3-6, a first fixed shaft insertion hole 30 is provided at the center of the blade 3 for inserting the fixed shaft 4, a support mounting groove 31 is provided in the first fixed shaft insertion hole 30 at the rotation center of the fan blade 3, a bearing support 6 is mounted inside the support mounting groove 31, the bearing support 6 is provided with a second fixed shaft insertion hole 60 for the fixed shaft 4 to be inserted therethrough, and a bearing limiting groove 61 is provided within the second fixed shaft insertion hole 60. A shaft shoulder 40 is provided on a shaft segment of the fixed shaft 4, and the bearing 5 abuts against the shaft shoulder 40 when mounted on the fixed shaft 4. During mounting, first, the bearing 5 is mounted in the bearing limiting groove 61, then the bearing support 6 mounted with the bearing 5 is mounted within the support mounting groove 31 of the fan blade 3, and finally, the fixed shaft 4 passes through the second fixed shaft insertion hole 60, the bearing 5 and the first fixed shaft insertion hole 30 in sequence. Through the above steps, the fan blade 3 can rotate relative to the fixed shaft 4 by means of the bearing 5, and the bearing 5 is provided at the rotation center of the fan blade 3, so that the bearing and the fan blade are in one plane, which is conducive to reduce the shaking amplitude of the fan blade, so that the fan blade can rotate more smoothly and reduce noise.

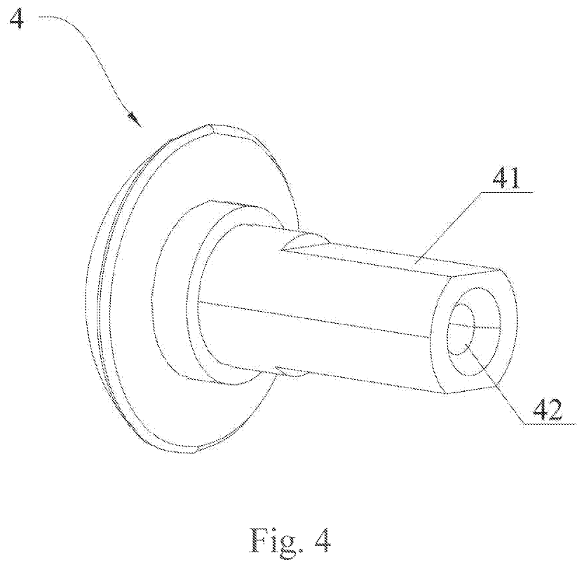

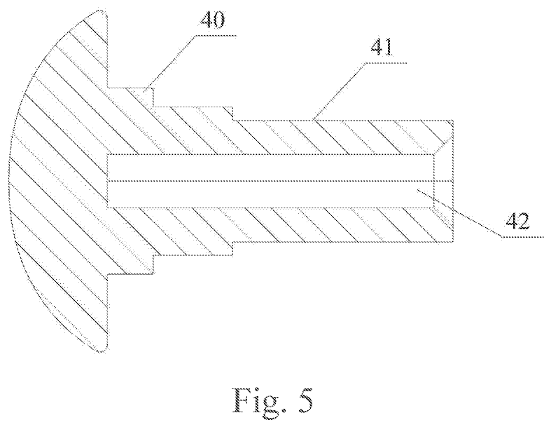

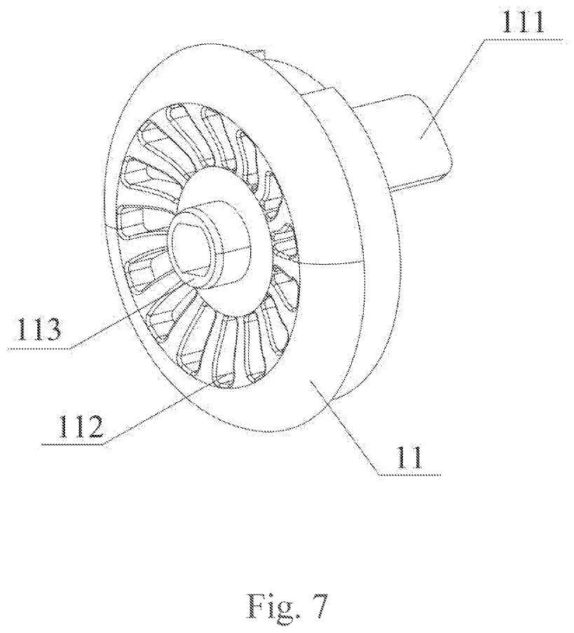

As shown in FIG. 4 and FIG. 5, a limiting shaft segment 41 is provided on one end of the fixed shaft 4 passing out of the fan blade 3 and is used for limiting connection with the front end housing 11. Correspondingly, as shown in FIG. 7 to FIG. 9, a shaft base 13 is provided at a center of the first ventilation structure 112 on the front end housing 11, a shaft limiting hole 131 having a cross section shape as the same as that of the limiting shaft segment 41 is provided on one end of the shaft base 13, so that when the fixed shaft 4 is inserted into the shaft inserting hole 131, the rotation between the fixed shaft 4 and the front end housing 11 is limited. A screw hole 42 is further provided on the end of the limiting shaft segment 41. Correspondingly, a screw through hole 132 in communication with the shaft limiting hole 131 is provided on the other end of the shaft base 13, and screws are sequentially screwed into the screw through hole 132 and the screw hole 42 to fixedly connect the fixed shaft 4 and the front end housing 11. Hence, the rotation of the fan blade 3 relative to the front end housing 11 is achieved.

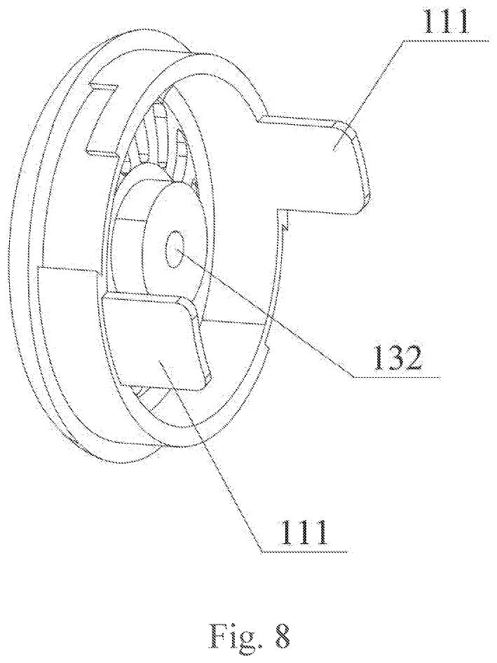

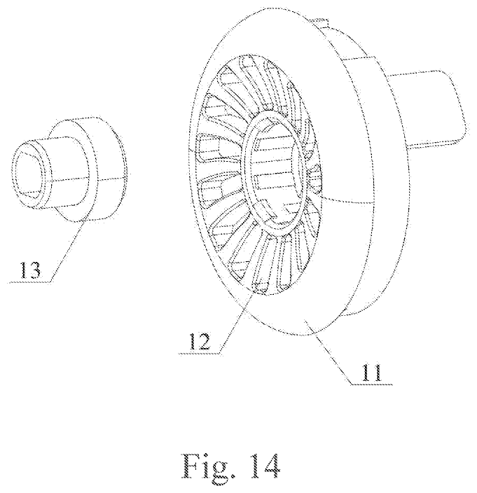

The shaft base 13 is integrally formed with or detachably fixedly connected to the front end housing 11. As shown in FIGS. 7 and 8, the shaft base 13 is integrally formed with the front end housing 11. As shown in FIG. 14, the shaft base 13 is fixedly connected to the front end housing 11 by means of a limiting groove provided at the center of the front end housing 11.

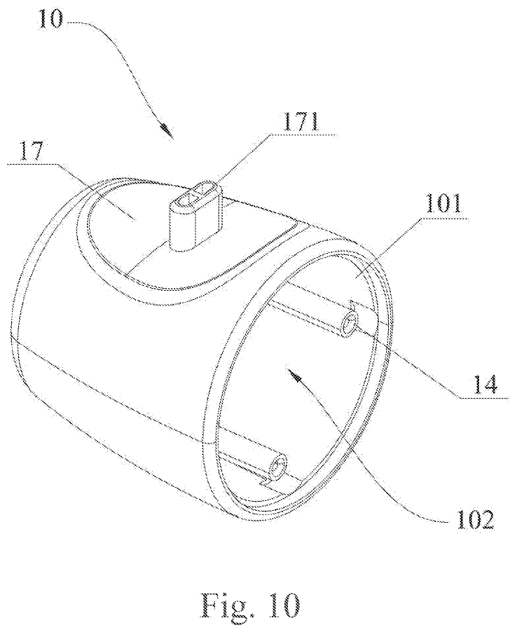

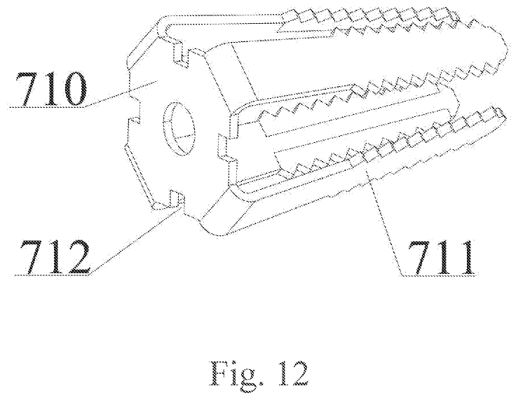

As shown in FIG. 10, three bolt fixing columns 14 are provided inside the main body 1, and are used for mounting a fixing plate 15 at the air inlet 101. Three mounting holes 151 fitted with the bolt fixing columns 14 are provided on the fixing plate 15, and the fixing plate 15 is fixedly connected to the main body 1 by bolts. A second ventilation structure 152 in communication with the first ventilation structure 112 is provided on the edge of the plate surface of the fixing plate 15 in the circumferential direction. A fixing member 7 is fixedly connected to the other side of the fixing plate 15 which is in contact with the main body 1, and is used for fixedly connecting the main body 1 to a grid at the air outlet of an air conditioner of an automobile. The fixing member 7 comprises a clamping claw 71. As shown in FIG. 12, the clamping claw 71 comprises a fixing sheet 710 used for being fixedly connected to the fixing plate 15 in a non-rotatable manner. Specifically, two limiting blocks 153 are further provided on the fixing plate 15, two limiting grooves 712 are provided on the fixing sheet 710 at positions corresponding to the limiting blocks 153, and the fixing sheet 710 is fixedly connected to the fixing plate 15 by means of screws after being fixedly mounted. Four claw sheets 711 extending from the edge of the fixing sheet 710, and a clamping claw sleeve 72 is sleeved on the claw sheets 711 and is used for fixing the clamping claw 71 to an external object in an anti-slip manner.

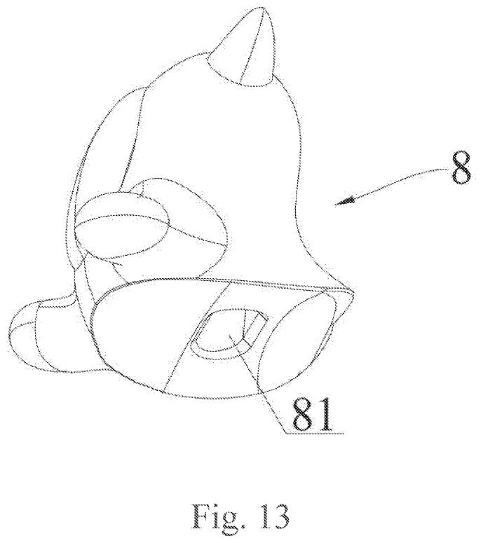

In order to improve the interestingness of the aromatherapy device, a doll 8 is further fixedly connected to the outer side of the main body 1. Specifically, a doll bottom plate 17 is provided on the main body 1, and a plug 171 is provided on the doll bottom plate 17. Correspondingly, as shown in FIG. 13, an insertion hole 81 is provided at the bottom of the doll 8, and the insertion hole 81 is in interference fit with the plug 171.

The perfume device 2 comprises a perfume sheet 20. the perfume sheet 20 is mounted inside the main body 1 and volatilizes fragrance outwards, and the volatilized fragrance gas is blown into the automobile through the air flow between the first ventilation structure 112 and the second ventilation structure 152 at both ends of the main body 1, making the diffusion range larger. In order to facilitate the fixing of the perfume sheet 20, as shown in FIG. 8, extension plates 111 arranged symmetrically is arranged on one side of the front end housing 11 fixedly connected to the main body 1, the perfume sheet 20 is clamped between the two extension plates 111, so that the perfume sheet 20 vertically stands in the air circulation channel between the first ventilation structure 112 and the second ventilation structure 152, facilitating the fragrance of the perfume sheet 20 to be volatile and stable; and when the device is in use, the front end housing 11 can be removed from the main body 1 to replace the perfume sheet 20, having a more convenient replacement process, and being unnecessary to remove the aromatherapy device from the grid of an air conditioner of the automobile as a whole.

### Second Embodiment

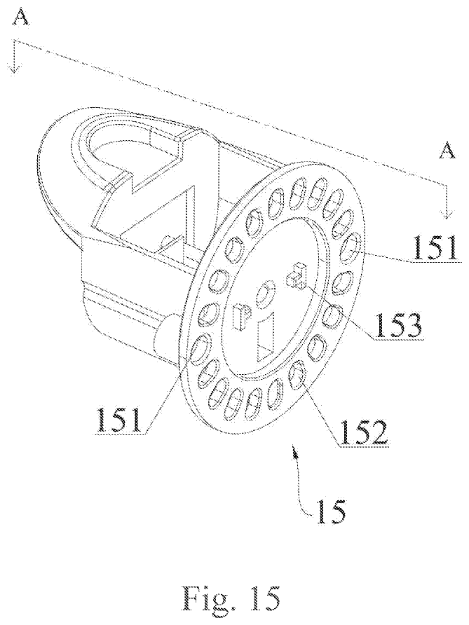

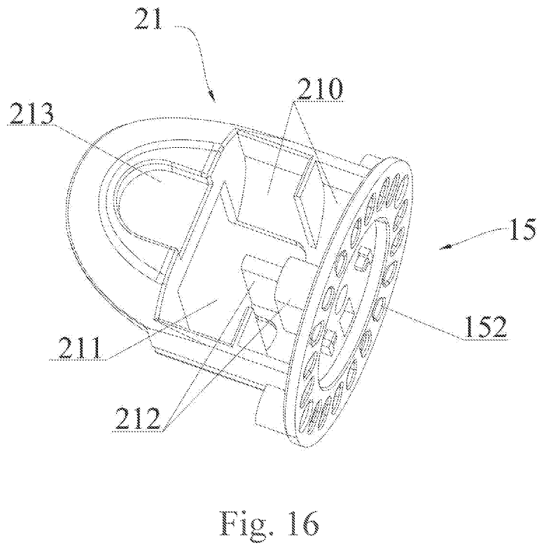

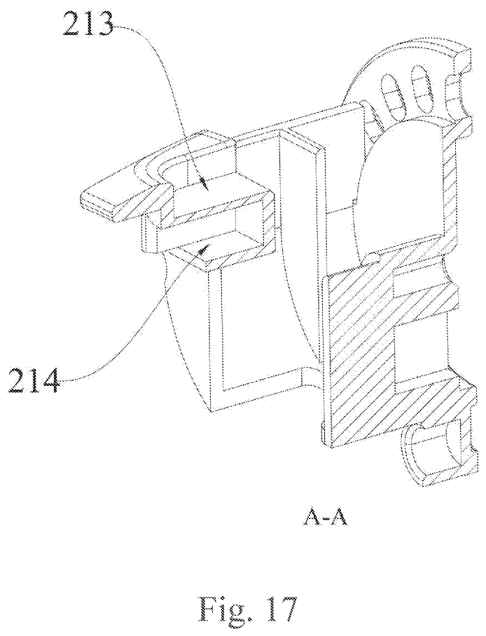

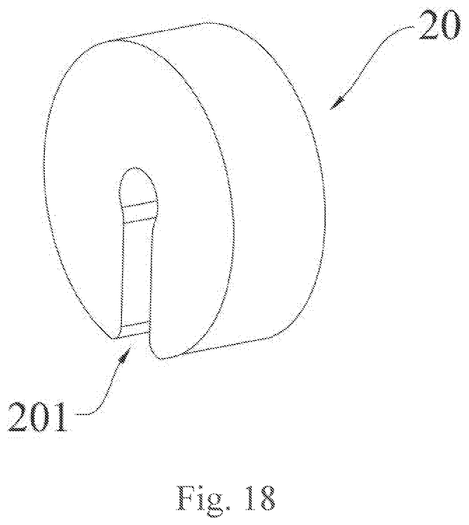

In order to facilitate the stable mounting of the perfume sheet 20, the present invention also provides a perfume accommodating box 21, which replaces the extension plates 111 provided on the front end housing 11 and makes the mounting more stable. As shown in FIGS. 15 to 18, the perfume accommodating box is fixedly connected to or integrally formed with the fixing plate 15, and is mounted in the cavity of the main body 1. Two perfume accommodating chambers 210 having an opening at the upper end are provided in the perfume accommodating box 21; a partition is provided between the two perfume accommodating chambers 210, and is used for filling a perfume sheet 20; a fragrance release port 211 is further provided at one end of a perfume accommodating chamber 210, and is used for releasing the fragrance volatilized from the perfume sheet 20. Further, the opening of the fragrance release port 211 can extend towards the bottom of the perfume accommodating chamber 210, increasing the contact area of the perfume sheet 20 and air, facilitating the release of fragrance. A perfume sheet limiting plate 212 is further provided on the bottom of the perfume accommodating chamber 210, correspondingly, the perfume sheet 20 is provided with a perfume sheet limiting groove 201 fitted with the perfume sheet limiting plate 212, achieving the further limitation to the position of the perfume sheet 20, causing that the perfume sheet 20 is more stably mounted, and preventing the old perfume sheet from being removed conveniently due to the dumping or shifting of the perfume sheet 20 in the perfume accommodating chamber 210. The limiting placement of the fragrance sheet 20 is more conducive to replacement operations.

### Third Embodiment

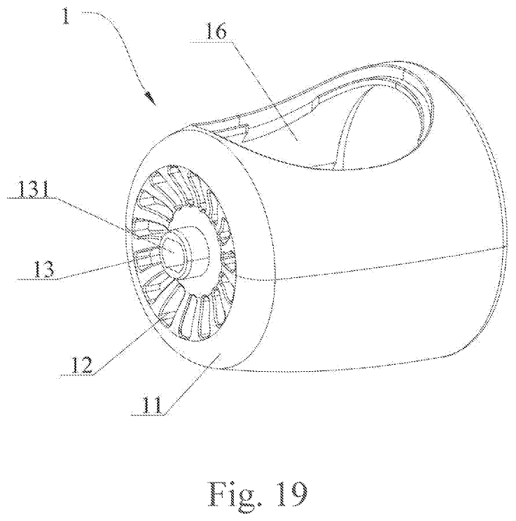

In order to facilitate the replacement of the perfume sheet 20 in the second embodiment, the present invention further provides a main body 1 having a material charging port 16. As shown in FIG. 19, the material charging port 16 is provided on a side edge of the main body 1 and is for replacing the perfume sheet 20 on the side edge, and the main body 1 does not need to be removed from a grid of an air conditioner of an automobile, thereby facilitating replacement. In addition, the doll bottom plate 17 is detachably connected to the main body 1, and the doll bottom plate 17 can be removed and replaced when replacing the perfume sheet 20 at the material charging port 16 of the doll bottom plate 17, being convenient and fast. In order to further stably mount the doll bottom plate 17 and the main body 1 and to facilitate the replacement of the perfume sheet 20, a first magnet mounting groove 172 is recessed downwards from the surface of the doll bottom plate 17, and a first magnet 18 is fixedly mounted inside the first magnet mounting groove 172, and correspondingly, a doll bottom plate positioning groove 213 is recessed downwards from the upper surface of the perfume accommodating box 21 and is used for accommodating the first magnet mounting groove 172, a second magnet mounting groove 214 is arranged directly below the doll bottom plate positioning groove 213, and a second magnet 214 is fixedly mounted inside the second magnet mounting groove 22. When the doll bottom plate 17 covers the material charging port 16, the first magnet 18 and the second magnet 22 attract each other, facilitating the fastening of the doll bottom plate 17, having a stable fastening, and preventing the inner perfume sheet 20 from falling due to the falling of the doll bottom plate 17 when the automobile jounces.

As the perfume sheet 20 does not need to be mounted on the front end housing 11, the front end housing 11 is integrally formed with the main body 1, facilitating processing.

### Fourth Embodiment

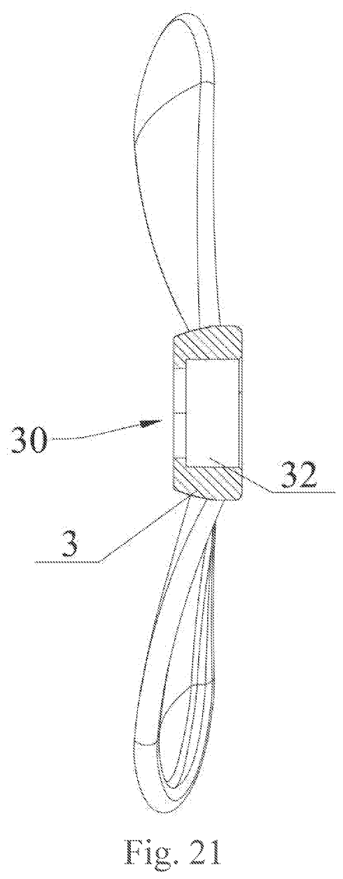

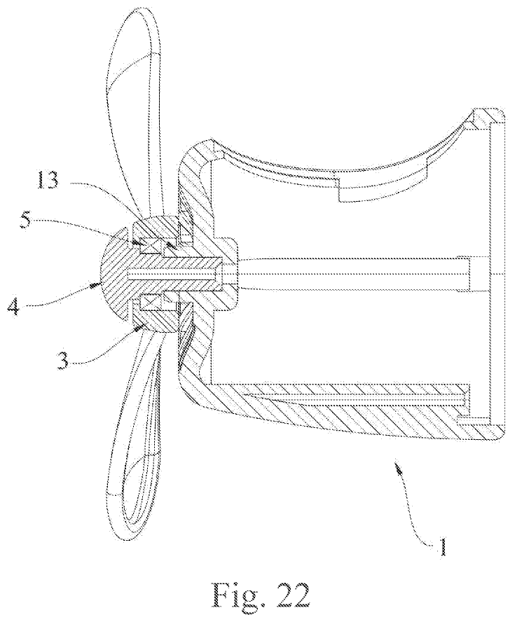

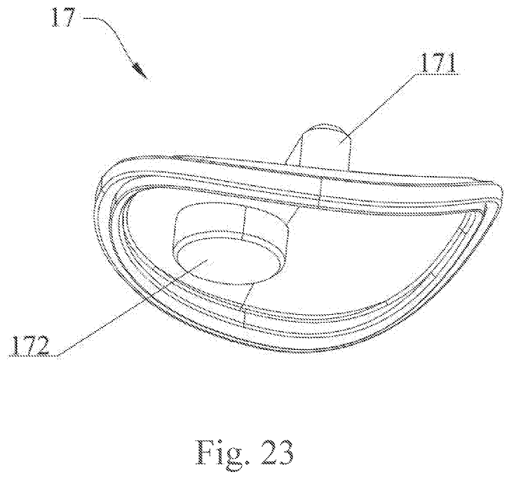

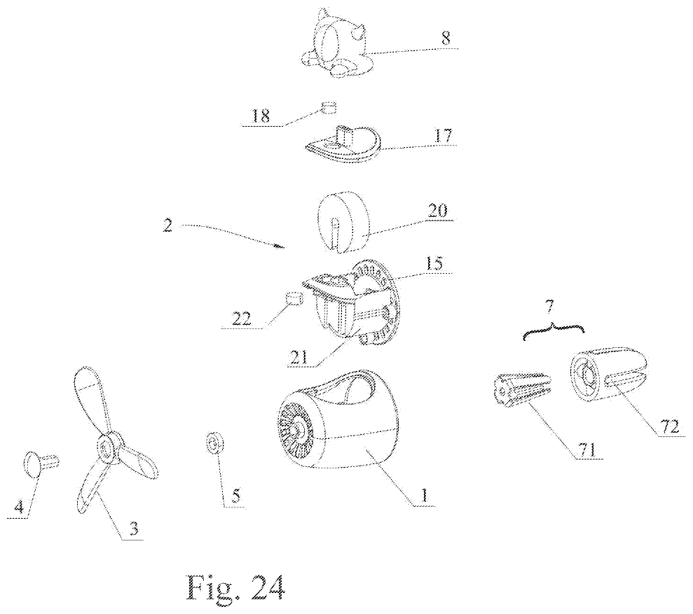

The present invention also provides a fan blade 3 without a bearing support 6. As shown in FIGS. 21 to 24, a bearing mounting groove 32 is provided on a side of a first fixed shaft insertion hole 30 of the fan blade 3 adjacent to a shaft base 13; and during mounting, a bearing 5 is mounted in the bearing mounting groove 32, and then a fixed shaft 4 passes through the first fixed shaft insertion hole 30 and the bearing 5 is fixedly connected to the shaft base 13. As the number of parts is reduced, the processing is more convenient, and the processing and mounting costs are reduced.

From the above description, it can be determined that the above embodiments of the present invention achieve the following technical effects:

1. According to the present invention, a main body of an aromatherapy machine in which an aromatherapy device is mounted is fixed at an air outlet of an air conditioner of an automobile by means of a fixing member, the air conditioner of the automobile is used to blow air to diffuse the fragrance into the automobile better, and the mounting is stable, preventing the phenomenon that the vehicle is unstable in fixed conditions such as jolt, sharp acceleration or deceleration.

2. In the present invention, a solid perfume sheet is mounted inside the vehicle-mounted aromatherapy machine, and the perfume sheet can be replaced by removing a front end housing; and the machine body part of the vehicle-mounted aromatherapy machine can be used for a long time, reducing the waste of resources.

3. In the present invention, a material charging port is provided on a side edge of a main housing, and a doll bottom plate is connected to the material charging port by means of magnetic attraction, and the doll bottom plate is removed from the material charging port or covered on the material charging port, so as to facilitate the replacement of perfume.

4. A doll is provided on the doll bottom plate, so that the vehicle-mounted aromatherapy machine as a whole has interestingness; when the doll bottom plate is removed from the material charging port, the doll can serve as a point of exertion, facilitating removal of the doll bottom plate.

5. A perfume accommodating box is mounted in a vehicle-mounted aromatherapy machine, facilitating the stabilization of a perfume sheet; a perfume sheet limiting plate is further provided in the perfume accommodating chamber thereof, and a perfume sheet limiting groove is provided on the corresponding perfume sheet to further define the position of the perfume sheet inside the aromatherapy machine, causing that the perfume sheet 20 is more stably mounted, and preventing the old perfume sheet from being removed conveniently due to the dumping or shifting of the perfume sheet 20 in the perfume accommodating chamber 210. The limiting placement of the fragrance sheet 20 is more conducive to replacement operations.

6. The vehicle-mounted aromatherapy machine is further provided with a fan blade for guiding flow, and the fan blade rotates under the action of blowing of an air-conditioner, so that the fragrance volatilized inside the aromatherapy machine is guided, facilitating the diffusion of the fragrance, and making the whole more interesting.

Compared with the prior art, the vehicle-mounted aromatherapy device of the present invention is mounted at an air outlet of an air conditioner of an automobile and is used for the volatilization and diffusion of solid perfume, avoiding the occurrence of the phenomenon of falling due to instability during the travel of the vehicle; and the vehicle-mounted aromatherapy device of the present invention can replace a perfume sheet, instead of a disposable product, reducing the waste of resources; and in the vehicle-mounted aromatherapy device of the present invention has a simple and convenient method for replacing a fragrance sheet and more stable mounting of the fragrance sheet inside, and the fragrance volatilizes more evenly.

The above are only the preferred embodiments of the present invention, and are not intended to limit the present invention. For a person skilled in the art, the present invention can have various modifications and variations. Any modifications, equivalent replacements, improvements and the like made within the spirit and principle of the present invention shall belong to the protection scope of the present invention.

1. A vehicle-mounted aromatherapy device, comprising:
a main body, wherein an accommodating cavity is provided inside the main body, the main body comprises a first ventilation structure and a second ventilation structure, the second ventilation structure is provided at one side of the accommodating cavity and is correspondingly mounted at an air outlet of an indoor air conditioner for air intake, the first ventilation structure is provided on the other side of the accommodating cavity, the first ventilation structure is used for air outflow of the vehicle-mounted aromatherapy device, and the first ventilation structure and the second ventilation structure are both in communication with the accommodating cavity;
a perfume device mounted in the accommodating cavity of the main body;
a fan blade mounted on a lateral exterior of the first ventilation structure of the main body, wherein the fan blade is rotatable relative to the main body; and
a fixing member fixedly connected to the main body, wherein the fixing member is capable of fixedly connecting the main body to an external object.

2. The vehicle-mounted aromatherapy device of claim 1,
wherein the main body comprises a main housing and a front end housing, an air outlet is provided at one end of the main housing, and the front end housing is provided on the air outlet and is detachably connected to the main housing.

3. The vehicle-mounted aromatherapy device of claim 2,
wherein an air inlet is provided at the other end of the main housing, the main body further comprises a fixing plate, the fixing plate is mounted on the air inlet of the main housing, and the second ventilation structure is provided on an outer surface of the fixing plate.

4. The vehicle-mounted aromatherapy device of claim 2,
wherein a doll bottom plate is provided on an outer wall of the main housing, and the doll bottom plate is integrally formed with or fixedly connected to the main housing.

5. The vehicle-mounted aromatherapy device of claim 2,
wherein two extension plates are symmetrically provided on an inner side of the front end housing, and are used for mounting the perfume device.

6. The vehicle-mounted aromatherapy device of claim 2,
wherein a shaft base is axially provided on the front end housing, the fan blade is connected to the shaft base by means of a fixed shaft, and the fan blade is rotatable relative to the fixed shaft.

7. The vehicle-mounted aromatherapy device of claim 6,
wherein a first fixed shaft insertion hole is provided in the center of the fan blade, a support mounting groove is provided in the first fixed shaft insertion hole at the rotation center of the fan blade, and a bearing support is mounted inside the support mounting groove;
a second fixed shaft insertion hole used for the fixed shaft to be inserted therethrough is provided on the bearing support, a bearing limiting groove is provided in the second fixed shaft insertion hole, and a bearing is mounted inside the bearing limiting groove;
the fixed shaft passes through the bearing support, the bearing and the fan blade in sequence, so as to be connected to the shaft base.

8. The vehicle-mounted aromatherapy device of claim 6,
wherein a first fixed shaft insertion hole is provided at the center of the fan blade, and a bearing mounting groove for mounting the bearing is provided in the first fixed shaft insertion hole; and the fixed shaft passes through the bearing and the fan blade in sequence, so as to be connected to the shaft base.

9. The vehicle-mounted aromatherapy device of claim 3,
wherein the vehicle-mounted aromatherapy device further comprises a doll, the doll is provided on an outer wall of the main body, and the doll is integrally formed with or fixedly connected to the main body.

10. The vehicle-mounted aromatherapy device of claim 2,
wherein the perfume device comprises a perfume accommodating box, and at least one perfume accommodating chamber is provided in the perfume accommodating box;
a fragrance release port is further provided on one end of the perfume accommodating chamber, and is used for releasing the fragrance volatilized by an internal perfume.

11. The vehicle-mounted aromatherapy device of claim 10,
wherein a perfume sheet is mounted inside the perfume accommodating chamber, a perfume sheet limiting plate is further provided at the bottom of the perfume accommodating chamber, and correspondingly, a perfume sheet limiting groove fitted with the perfume sheet limiting plate is provided on the perfume sheet.

12. The vehicle-mounted aromatherapy device of claim 10,
wherein the front end housing is integrally formed with the main housing, a material charging port is provided on a side surface of the main housing, the material charging port is opposite to the perfume accommodating chamber in position, and the doll bottom plate is detachably connected to the material charging port.

13. The vehicle-mounted aromatherapy device of claim 12,
wherein a first magnet mounting groove is recessed downwards from the surface of the doll bottom plate, a first magnet is fixedly mounted inside the first magnet mounting groove, correspondingly, a doll bottom plate positioning groove is recessed downwards from the upper surface of the perfume accommodating box and is used for accommodating the first magnet mounting groove, a second magnet mounting groove is arranged directly below the doll bottom plate positioning groove, and a second magnet is fixedly mounted inside the second magnet mounting groove.

14. The vehicle-mounted aromatherapy device of claim 4,
wherein the doll is integrally formed or fixedly mounted on an upper part of the doll bottom plate.

15. The vehicle-mounted aromatherapy device of claim 14,
wherein a doll plug is arranged above the doll bottom plate, and correspondingly, an insertion hole fitted with the doll plug is arranged at the bottom of the doll.

16. The vehicle-mounted aromatherapy device of claim 2,
wherein the fixing member comprises a clamping claw, and the clamping claw is fixedly connected to the fixing plate.

17. The vehicle-mounted aromatherapy device of claim 16,
wherein the clamping claw comprises a fixing sheet, and at least two claw sheets are arranged on an edge of the fixing sheet.

18. The vehicle-mounted aromatherapy device of claim 17,
wherein at least one limiting block is provided on the fixing plate,
correspondingly, a limiting groove is provided on the fixing sheet and is used to prevent relative rotation between the clamping claw and the fixing plate.

19. The vehicle-mounted aromatherapy device of claim 16,
wherein a clamping jaw sleeve is sleeved on the clamping jaw, so as to increase a friction force between the clamping jaw and the external object.

20. The vehicle-mounted aromatherapy device of claim 1,
wherein the main body comprises a main housing and a front end housing, an air outlet is provided at one end of the main housing, and the front end housing is provided on the air outlet and is integrally formed with the main housing.
